(19) United States
(12) Patent Application Publication
(10) Pub. No.: US 2006/0172922 A1
(43) Pub. Date: Aug. 3, 2006

(54) Methods for combating ischemic injury to epithelial organ

(75) Inventor: Sanjay Kumar Nigam, Del Mar, CA (US)

Correspondence Address: BUCHANAN INGERSOLL LLP;(INCLUDING BURNS, DOANE, SWECKER & MATHIS), P.O. BOX 1404, ALEXANDRIA, VA, 22313-1404, US

(73) Assignee: The Regents of the University of California, Oakland, CA (US)

(21) Appl. No.: 11/352,746

(22) Filed: Feb. 10, 2006

Related U.S. Application Data
(63) Continuation of application No. 09/965,651, filed on Sep. 25, 2001.
(60) Provisional application No. 60/235,387, filed on Sep. 25, 2000.

(51) Int. Cl. A61K 38/17; A61K 31/7034

(52) U.S. Cl. 514/2; 514/35

(57) ABSTRACT

A method for enhancing recovery by epithelial cells from ischemia by targeting distinct lesions. The method comprises inhibiting internalization of intercellular junctions, E-cadherin, occludin or other membrane proteins; promoting reuse of preexisting components by targeting for activation specific signaling events during short-term ischemia; inhibiting degradation of E-cadherin or other key proteins necessary for the maintenance of the polarized epithelial cell phenotype; and enhancing the protein folding and assembly capacity in the endoplasmic reticulum and/or cytosol with agents which upregulate cytoprotective chaperones, wherein the enhancing helps to reconstruct degraded adherens and tight junctions by de novo synthesis and movement of membrane proteins, and alleviation of cellular stress by raising levels of molecular chaperones.

### CROSS REFERENCE TO RELATED APPLICATION

This application is a continuation of U.S. patent application Ser. No. 09/965,651, filed Sep. 25, 2001, the disclosure of which is incorporated herein by reference in its entirety.

### GOVERNMENT INTERESTS

Research for this patent application was supported in part by grants to S. K. Nigam from the National Institute of Diabetes and Digestive and Kidney Diseases Nos. R01-DK53507 and R01-DK51211.

### FIELD OF THE INVENTION

The present invention generally concerns a new method for combating ischemic injury to epithelial organs.

The present invention particularly concerns a new comprehensive method and procedure for combating ischemic injury to epithelial organs using treatments targeted to specific status of ischemia and involving specific cell structures.

### DESCRIPTION OF THE RELATED ART

A major cause of morbidity and mortality comprises ischemic injury to predominantly epithelial organs such as the kidney. [Kevin T. Bush, Steven H. Keller, and Sanjay K. Nigam, Genesis and reversal of the ischemic phenotype in epithelial cells. J. Clin. Invest. 106:621-625-incorporated herein by reference]. For example, it is estimated that around 50% of all cases in hospitalized patients with acute renal failure are ischemic in origin (1). Regardless of severity of the situation, progress in the medical management of this and other syndromes in which ischemia occurs has advanced at a snail's pace. This may be attributed, in part, to the merely rudimentary understanding of the cell biology underlying the ischemic epithelial phenotype, and the molecular mechanisms behind the recovery of normal cell and tissue organization.

Besides providing a physical barrier between biologic compartments, kidney, gut and other epithelial tissues also mediate vectorial and selective transport of ions, water, and macromolecules between blood and the external environment. These various functions depend on the integrity of intercellular junctions, the arrangement of lipids and proteins in the plasmamembrane into strictly maintained apical and basolateral domains, and productive cell-substratum interactions, all of which are severely affected by ischemia.

Although other factors, such as oxidative damage and ion and pH changes, likely play important roles in the generation of the ischemic epithelial tissue damage, the predominate cause is believed to be depletion of cellular ATP (2,3). Cell culture models, using agents that deplete cellular ATP, have been used extensively to study ischemic injury in polarized epithelial cells (3). Although the fidelity of the lesions produced in such models to those observed in vivo has been debated, there is little doubt that these ATP depletion/repletion cell culture models provide valuable insights into the molecular mechanisms underlying ischemic injury and recovery. This is supported by the observation of similar cellular and molecular lesions in cells of the ischemic whole organ. Many of these lesions appear to be remarkably specific, biochemically identifiable, and likely regulated. Recovery after short-term injury appears to be mediated by a multifactoral combination of previously elucidated and novel sorting mechanisms transduced by “classical” signaling pathways.

Some of the other known cellular and molecular lesions induced by ischemia and/or ATP depletion include: misfolding and/or aggregation of membrane and secreted proteins (4); disruption of the actin-based cytoskeleton (5); disturbances in apical-basolateral protein polarization (6); mislocalization and degradation of protein components of the intercellular junctions (7, 8); upregulation of a number of genes, including molecular chaperones (4, 9), growth factors and their receptors (10); perturbation of integrin-mediated cell-substratum adhesion (11-13); and induction of programmed and nonprogrammed cell death (2). Alterations in the actin cytoskeleton and integrin-mediated cell-substratum interactions have been extensively reviewed elsewhere (5, 13). Herein, the focus is primarily on recent information related to lesions affecting the permeability barrier, signaling events involved in the recovery of this barrier, and the roles of molecular chaperones in protecting epithelial cells. It would be extremely advantageous to combine a multifaceted approach to treatment of the above defined lesions by marshalling the individually described events into an encopassing treatment regimen.

### SUMMARY OF THE INVENTION

The primary object of this invention is to provide a comprehensive method for enhancing recovery by epithelial cells from ischemia and/or ATP depletion.

Another object in accordance with the present invention is to treat the ischemic lesion at various stages of progression of the disease.

Another object of this invention is to target the treatments for ischemia to precise molecular moieties and cellular locations.

A further, most preferred object is to provide a precise specific treatment strategy targeted toward inducing the damaged epithelial cells to reuse undamaged molecules and structures, to repair damaged molecules and structures, and to synthesize de novo the required molecules and structures.

In accordance with these objects, this invention contemplates a comprehensive method for enhancing recovery by epithelial cells from ischemia by targeting distinct lesions. The method involves inhibiting internalization of intercellular junctions, E-cadherin, occludin or other membrane proteins. The inhibiting of the internalization requires early intervention with drugs or growth factors that specifically modulate signaling through IP₃-sensitive calcium stores, G-proteins, protein kinase C, and other kinases all of which are implicated in the reassembly response during the calcium switch.

These include insulin-like growth factors, pleiotrophin, midkine, fibroblast growth factors, epidermal growth factor receptor ligands, melanocyte stimulating hormone, hepatocyte growth factor and related growth factors) that specifically modulate signaling through IP3-sensitive calcium stores (eg. nonhydrolyzable and other IP3 analogs), phosphoinositol-3-kinase (eg. specific activators), protein kinase C (eg. diacylglycerol analogs and other activators), small and large GTP binding proteins (eg. cell permeant nonhydrolyzable GTP analogs, aluminum fluoride, lysophosphatidic acid, phenylephrine), tyrsoine kinases (eg. specific activators).

The contemplated method further involves promoting reuse of preexisting components by targeting for activation specific signaling events during short-term ischemia, The promoting refers to facilitating the resorting of growth factor receptors to the cell surface through modulation of signaling pathways to enhance the effectiveness of endogenous and/or exogenous growth factors administered after ischemic insult.

Examples include treatment with other growth factors such as those mentioned above, protein kinase C activators such as diacylglycerol analogs; activators of small and large GTP binding proteins such as cell permeant nonhydrolyzable GTP analogs, aluminum fluoride, lysophosphatidic acid and phenylephrine; tyrosine kinase activators; activators of other kinases) through modulation of signaling pathways to enhance the effectiveness of endogenous and/or exogenous growth factors administered after ischemic insult and/or other types of injury.

A more specific and preferred embodiment of this invention is a method for inhibiting degradation of E-cadherin or other key proteins necessary for the maintenance of the polarized epithelial cell phenotype. In one embodiment, the inhibiting of degradation refers to prevention of proteolytic clipping of key proteins. For example, proteasome inhibitors such as MG 132 and lactocystin; and/or inhibitors of caspases and compounds with similar effects; and/or treatment with growth factors such as those mentioned above.

A most preferred embodiment in accordance with this invention is a method for enhancing the protein folding and assembly capacity in the endoplasmic reticulum and/or cytosol with agents that upregulate cytoprotective chaperones, wherein the enhancing helps to reconstruct degraded adherens and tight junctions by de novo synthesis and movement of membrane proteins, and alleviation of cellular stress by raising levels of molecular chaperones. In one embodiment, the agents which upregulate cytoprotective chaperones comprise inhibitors of proteasome, such as lactacystin, MG132 and others.

In another embodiment, the agents which upregulate cytoprotective cytosolic, endoplasmic reticulum and other chaperones comprises pretreatment with inducers of mild heat shock or a stress response (eg. proteasome inhibitors such as MG 132 and lactocystin and compounds with similar activity; and inducers of endoplasmic reticulum stress responses such as tunicamycin, geldanomycin and compounds with similar effects on the stress response.)

Still further embodiments and advantages of the invention will become apparent to those skilled in the art upon reading the entire disclosure contained herein.

### BRIEF DESCRIPTION OF THE DRAWING FIGURES

FIG. 1: A schematic representation of the methodology and salient points of this invention depicting general aspects of epithelial cell recovery after ischemia or ATP depletion. The ability of the cell to recover is dependent on the duration and extent of the ischemic insult and can be described as: (a) short-term and/or modest ischemia, (b) intermediate ischemia, and (c) prolonged and/or severe ischemia. After short-term and/or modest ischemia, degradation of critical junctional components has yet to occur, and cells can reestablish the tight, polarized epithelial cell phenotype primarily by reusing existing junctional components (e.g., E-cadherin, catenins) that have been internalized. As described in the text, this may require activation of signaling pathways involving tyrosine phosphorylation, calcium, and GTP. Intermediate ischemia is characterized by the beginning of the degradation of some of the junctional components (e.g., E-cadherin), and complete recovery from such an insult would likely involve a combination of reutilization of existing components together with synthesis of new junctional components. In the case of prolonged and/or severe ischemic injury, degradation of junctional components has proceeded to such an extent that recovery depends primarily on synthesis and assembly of new junctional macromolecular complexes, key proteins of which are folded in the ER. If the ischemic insult is not removed at this point, cell death (either apoptotic or necrotic) will ultimately be the result. The hypothesized relative importance of various pathways under each scenario is indicted by the thickness of the arrows. Intracellular junctions, such as the AJ, serve as an example, but other damaged cellular components may also become more dependent on de novo protein synthesis and ER folding/assembly for recovery as the length or severity of the ischemic insult increases.

### DESCRIPTION OF THE PREFERRED EMBODIMENT

1. Introduction

Homotypic interactions of the extracellular domains of multiple transmembrane adhesion molecules between adjacent cells establish and maintain a selectively permeable barrier. Such molecules include E-cadherin in the adherens junction (AJ) and the occludin/claudin families in the tight junction (TJ). The intracellular domains of these adhesion molecules also interact (directly or indirectly) with a number of cytoplasmic proteins, including, α., β, and γ catenin in the AJ, and zonula occludens-1 (ZO-1), ZO-2, ZO-3, and fodrin in the TJ, providing a functional link to the actin-based cytoskeleton. These interactions also modulate the stability of the adhesion proteins either by maintaining their appropriate conformations to recognize extracellular domains in adjoining cells or perhaps by inhibiting internalization and degradation. Under ischemic conditions, it appears that many of these cellular processes/structures are compromised, promoting junctional protein internalization and degradation, thereby disturbing the cell-cell interactions and the permeability barrier. Identifying molecular mechanisms underlying the cascade of events that induce cellular injury and those involved in the cell's recovery is key to developing rational therapeutic approaches to diminish the morbidity associated with ischemic injury to epithelial tissues. Many of these mechanisms were first elucidated by the inventors and have been exclusively characterized by them. Several of these are described below.

The Adherens Junction

In cell culture models, polarization and intercellular junctions depend in large part on cell-cell contact mediated by E-cadherin and subsequent assembly of the AJ. For example, treatment of polarizing epithelial cells with anti-E-cadherin antibodies disrupts junction assembly and retards the generation of the polarized epithelial phenotype (14). Alternatively, transfection of E-cadherin into nonpolarized fibroblasts induces a polarized distribution of NaKATPase somewhat akin to that seen in polarized epithelial cells (15). In addition, the cadherin-catenin interactions within the AJ are also critical to the formation and maintenance of the polarized epithelia (16).

ATP depletion of cultured renal epithelial cells results in rapid internalization of E-cadherin (17). Even under normal physiological conditions, E-cadherin is selectively internalized and recycled to the cell surface in a clathrin-mediated recycling endosomal pathway (18); it remains to be determined whether this or another pathway is involved in internalization and re-sorting of E-cadherin after ischemia. A somewhat more prolonged insult leads not only to internalization of E-cadherin, but also to proteolytic clipping of this protein at a specific site and to the disruption of normal cadherin-catenin interactions (8). Identification of the site of E-cadherin cleavage as well as the protease involved will shed considerable mechanistic light on the disruption of the AJ in ischemia. Interestingly, although E-cadherin itself is cleaved, its cytoplasmic binding partners the catenins remain near their steady-state levels for prolonged periods of ATP depletion (8).

Because functional AJs are critical for the establishment and maintenance of tight polarized epithelia (including TJ formation and polarized sorting of membrane proteins), degradation of E-cadherin, as well as disruption of cadherin-catenin interactions, likely constitutes a critical lesion in epithelial is ischemia. Over the long term, reassembly of the AJ in recovering epithelial tissue must depend on resynthesis of E-cadherin, assembly with the catenins, and reformation of functional AJs. How this occurs remains unclear, although it is possible that the undegraded catenins are recruited from the cytoplasm and reassembled with de novo synthesized E-cadherin at the endoplasmic reticulum (ER) itself or at a more distal compartment in the secretory pathway, after which they may be targeted to the cell surface to help reconstruct the AJ. Repair of more permanent AJ structures might depend on turnover of the proteins exposed to ischemic injury and on the de novo synthesis and assembly of new components. As discussed hereinbelow, supra, a limiting factor in the face of sustained ischemia may be the inability of the ER to fold newly synthesized membrane and secreted proteins such as E-cadherin (4).

The Tight Junction

The most apically positioned junction delineating the apical and basolateral surfaces of the epithelial cell, the TJ prevents diffusion of lipids in the membrane between the apical to basolateral surfaces. Its component molecules form the physical permeability barrier to solutes and liquids. The TJ comprises transmembrane proteins, the occludins and claudins (19), which are probably linked to the cytoskeleton through interactions with cytoplasmic proteins, including the zonula occludens (ZO-1, ZO-2, and ZO-3) and actin-binding proteins, such as fodrin (20).

Occludin is internalized and becomes associated with large insoluble complexes of ZO-1 and fodrin (7) in cell culture models of ischemia. After brief periods of ATP depletion, and recovery in the presence of ATP, these junctional components appear to redistribute promptly to their former locations. Prolonged and severe ATP depletion may, however, marshal the junctional proteins into the cellular degradative pathway. Therefore, after prolonged injury, repair must take place by de novo synthesis accompanied by movement of membrane proteins through the secretory pathway and reassembly with cytosolic components. The sorting and bioassembly pathways may be distinct from those thought to operate under normal physiological conditions.

A great deal of work has been done on the biogenesis of the TJ using the Madin-Darby canine kidney (MDCK) cell “calcium switch” model for TJ assembly (20), aspects of which resemble cell culture models of ischemia MDCK monolayers transferred to low calcium media lose cell to cell contacts, and internalize their intercellular junctions. They also suffer loss of apical and basolateral protein polarity, disruption of their actin cytoskeletons, and change in cell shape. Disruption of vectorial transport and loss of the permeability barrier result from these perturbations. However, switching back to normal calcium media induces cell to cell contact and restores the intercellular junctions, a normally configured cytoskeleton, a more columnar cell shape, and normal apical-basolateral polarity and barrier function. Studies of this model have implicated a number of signaling molecules associated with the reassembly of intercellular junctions, including protein kinase C, calcium, and heterotrimeric G proteins (20). Although there are important distinctions in the cellular biochemistry between the calcium-switch and ATP depletion/repletion model, recent studies have also implicated signaling pathways involving intracellular calcium, small GTP-binding proteins and tyrosine kinase activities in recovery of the epithelial cell phenotype after short-term ATP depletion (21-23). Indirect evidence suggests that certain signaling events modulate the rephosphorylation of TJ proteins, their release from cytoskeletal components, and perhaps dissolution of large macromolecular complexes and aggregates accumulating during ATP depletion (7, 22, 23). Also likely contribute to the protein processing involved in assembling and maintaining TJs are vesicular trafficking, endocytosis, and ubiquitination—all known to be modulated by cellular signaling.

Cellular Stress Responses and Cytoprotection

Ischemic conditions and ATP depletion are thought to promote the misfolding and/or denaturation of cellular proteins, either directly or through perturbation of their biosynthetic/folding pathways (9,24). Such impairment leads to a cellular stress response manifested by increases in the levels of mRNAs encoding the cytosolic stress proteins (e.g., the heat-shock proteins, including members of the Hsp70 family) (25), as well as the ER stress proteins (e.g., Grp78/BiP, Grp94, and ERp72) (4,24). These stress protein groups appear to function as molecular chaperones in the folding and assembly of proteins by temporarily stabilizing polypeptides, and thus preventing the occurrence of inappropriate intra- and intermolecular interactions and aggregation during the folding process (26). In the stress response, molecular chaperones are thought to be essential to cell survival through their ability to bind abnormal proteins and thereby prevent their aggregation. Most appear to depend on cellular ATP for their function (27).

Enhanced survival of cells subjected to a subsequent injury including ischemia/reperfusion and energy deprivation (ATP depletion) (28) correlate with elevated levels of cytosolic chaperones, especially members of the Hsp70 family. Although the exact mechanism of this cytoprotection remains to be filly elucidated, it is possible that the chaperoning activity protects cells by increasing refolding and limiting the potentially toxic aggregation of cellular proteins (29). Increased levels of the Hsps could also protect cells after more prolonged ischemia/reperfusion and/or ATP depletion/repletion by interfering with NF-B-mediated transcriptional activation of proinflammatory cytokine genes (30).

ER molecular chaperones may have similar cytoprotective properties. Upregulation of both cytosolic and ER molecular chaperones after treatment with inhibitors of the proteasome has been shown to protect epithelial cells subjected to thermal stress (31). Pretreatment with tunicamycin, an inhibitor of N-linked glycosylation that specifically induces accumulation of ER molecular chaperones, was found to enhance the survival of ATP-depleted renal epithelial cells in culture (9). These experiments indicate that ER chaperones alone can provide cytoprotection.

Therefore, as in the case of the cytosolic heat-shock proteins, overexpression of the ER molecular chaperones correlates positively with increased survival of cells subjected to ischemia/reperfusion (9). Although the mechanism of cytoprotection remains unclear, it is possible that enhanced cell survival is in part the result of increased chaperone function in the ER. On the other hand, because the ER serves as the major storage site of intracellular calcium, and several of the ER molecular chaperones bind calcium, induction of these proteins may help moderate the dramatic rises in cytosolic free calcium that occurs in ischemia or ATP depletion. Thus, the threat of oxidative stress to the cell is reduced (32-36).

Molecular Aspects of Epithelial Ischemia and Recovery: a Model

No central defect that can account for the various aspects of the ischemic epithelial phenotype has been found to date; however, recent work has revealed that the lesions of the ischemic epithelial cell are remarkably specific. These lesions can be defined in considerable biochemical detail, at least in cell culture models of ischemia. Equally remarkable is the ability of the injured kidney, as well as injured cells in culture, to recover their structure and function virtually completely, even when considerably damaged by ischemia or ATP depletion. This recovery appears to be largely dependent on the magnitude and the duration of the kidney ischemia. Renal tubules injured by sublethal ischemic insult fully recover and re-establish kidney function, whereas prolonged ischemia ultimately leads to cell death. This, in turn, can induce an inflammatory response greatly limiting the ability of the tubules to recover.

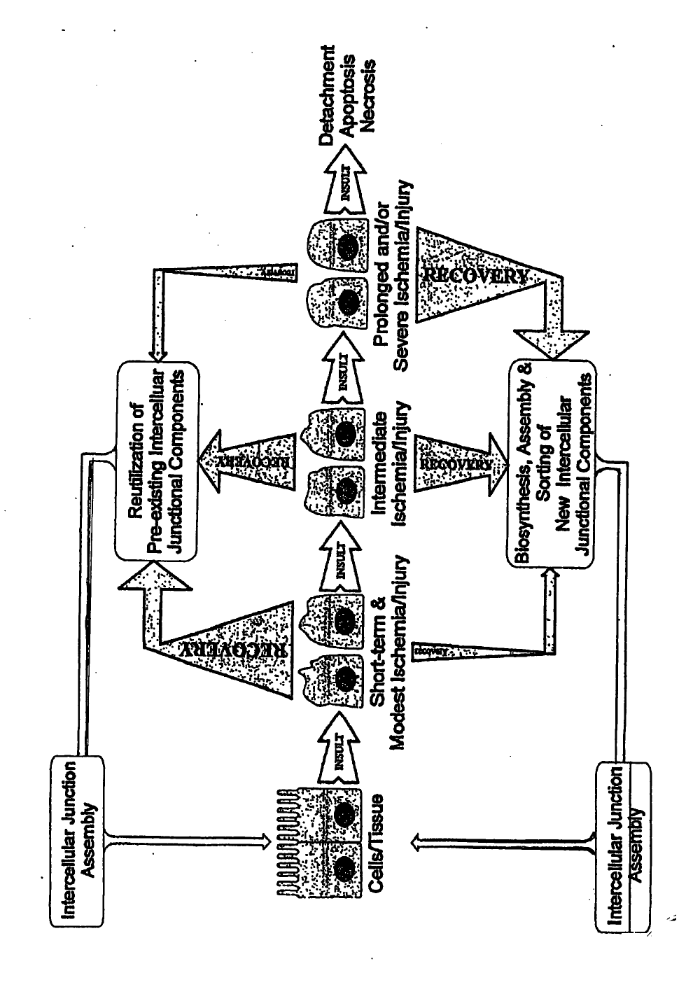

To better understand the molecular and cellular pathology of the ischemic epithelial phenotype, and mechanisms underlying its restoration to normalcy, it is important to distinguish among events leading to short-term and/or modest, intermediate, or prolonged and/or severe ischemic injury, as is shown in FIG. 1. Although the model shown focuses primarily on damage to multiprotein complexes in intercellular junctions, such as the AJ, similar consideration might apply to damage to other cell surface molecules and intracellular components.

Short-Term Ischemia

Although short-term ischemia causes the redistribution of cell-surface molecules and cytoskeletal disruption, it does not induce detectable loss of E-cadherin or other rapidly degraded molecules. Recovery of the tight polarized epithelial cell phenotype under these conditions is likely to depend on reusing existing components that became internalized, aggregated, or bound to the cytoskeleton during the ischemic period (7, 8, 17). This reassembly pathway likely depends on classical signaling pathways involving calcium (23), small GTP binding proteins (21), and tyrosine phosphorylation (22). This response may in fact be conceptually similar to the reassembly mechanisms elucidated using the MDCK calcium switch model, which depends solely on reuse of preexisting components. Therefore, early intervention with drugs or growth factors that specifically modulate signaling through IP.sub.3-sensitive calcium stores, G-proteins, protein kinase C, and other kinases—all of which are implicated in the reassembly response during the calcium switch—may enhance recovery and minimize injury (20, 22). It seems likely that at least some of the sorting and bioassembly pathways used by cells recovering from injury are distinct from those used under normal physiological conditions or in the calcium switch model. It is also worth noting that growth factor receptors may be internalized during ischemia, and the well-documented upregulation of growth factor receptors may be one response to this internalization (10). Facilitating the resorting of growth factor receptors to the cell surface through modulation of signaling pathways could enhance the effectiveness of endogenous and/or exogenous growth factors administered after ischemic insult.

Intermediate Stage of Ischemia

During the intermediate stage of ischemia, some components of intercellular junctions (e.g., E-cadherin) and perhaps other proteins are rapidly degraded, whereas other components (e.g., ZO-1, catenins) remain intact (8). However, as in short-term ischemia, many of these proteins become redistributed at the plasma membrane, internalized, found tightly associated with the actin-based cytoskeleton, or aggregated (7, 8). Recovery would depend on reuse of existing components through the action of classical signaling events involving calcium, GTP, and tyrosine phosphorylation, together with de novo synthesis of key degraded proteins (e.g., E-cadherin) and reassembly of macromolecular complexes. The rate-limiting step here could be assembly and folding within the endoplasmic reticulum, which itself is dysfunctional in the setting of ischemia (4, 24). Additional lesions may also exist elsewhere in the secretory pathway. Moreover, the final reassembly of multiprotein complexes, such as those that comprise the AJ, is likely to be quite different. The de novo synthesized E-cadherin that is translocated into the ER will presumably link to pre-existing catenins, which were not degraded after injury but that have moved into an as yet unidentified cell compartment. In the TJ, its constituent proteins appear to associate with a cytoplasmic membrane compartment, and the cytoskeleton and may also aggregate (7, 8).

Depending on the duration and severity of ischemic injury, a combination of such potentially novel reassembly pathway(s) and the normal physiological secretory pathway (beginning with the biosynthesis and maturation of membrane proteins in the ER) may be necessary to effectively restore structures like the AJ, on which the cell's polarized distribution of membrane proteins and the tissue's capacity to act as a permeability barrier both depend. Severe Ischemia After prolonged and severe, but still sublethal ischemic insult, many key membrane and secreted proteins (E-cadherin, claudins, occludins, integrins, matrix, molecules, and so forth) are degraded or targeted for more rapid degradation. In addition, the injured cell, or its concerned neighbor, is likely to make an attempt at repair through the elaboration of growth factors and cytokines that must likewise pass through the secretory pathway. Therefore, a rate-limiting step for repair is likely to be bioassembly and folding in the ER and subsequent sorting through the secretory pathway (24). However, in the setting of such severe ischemia, it is likely that the capacity of the ER to correct the misfolding/aggregation of secretory and membrane proteins through the action of ER molecular chaperones will be severely compromised (4). Some of the preexisting components that have not been degraded may still be useful, but the ultimate restoration of the polarized epithelial phenotype will require the biosynthesis and assembly of both secreted and cytosolic components of the crucial plasma membrane-associated complexes.

Therefore, strategies designed to enhance epithelial cell recovery may have to target several distinct lesions. First, therapies should be designed to inhibit the internalization and promote the reuse of preexisting components, perhaps by targeting specific signaling events. Second, it will be necessary to inhibit the degradation of E-cadherin or other key proteins necessary for the maintenance of the polarized epithelial cell phenotype. Third, effective treatment, particularly of severe ischemic injury, may require enhancing the protein folding and assembly capacity in the ER and/or cytosol with agents which upregulate cytoprotective chaperones.

While the present invention has now been described in terms of certain preferred embodiments, and exemplified with respect thereto, one skilled in the art will readily appreciate that various modifications, changes, omissions and substitutions may be made without departing from the spirit thereof. It is intended, therefore, that the present invention be limited solely by the scope of the following claims.

### REFERENCES

1. Thadhani, R., Pascual, M., and Bonventre, J. V. 1996. Acute renal failure. N. Engl. J. Med. 334:1448-1460.
2. Bonventre, J. et al. 1998. Acute renal failure. I. Relative importance of proximal vs. distal tubular injury. Am. J. Physiol. Renal Physiol. 275:F623-F631.
3. Molitoris, B. et al. 2000. Acute renal failure. II. Experimental models of acute renal failure: imperfect but indispensable. Am. J. Physiol. Renal Physiol. 278:F1-F12.
4. Kuznetsov, G., Bush, K. T., Zhang, P. L., and Nigam, S. K. 1996. Perturbations in maturation of secretory proteins and their association with endoplasmic reticulum chaperones in a cell culture model for epithelial ischemia. Proc. Natl. Acad. Sci. USA. 93:8584-8589.
5. Molitoris, B. A., Leiser, J., and Wagner, M. C. 1997. Role of the actin cytoskeleton in ischemia-induced cell injury and repair. Pediatr. Nephrol. 11:761-767.
6. Fish, E. M., and Molitoris, B. A. 1994. Alterations in epithelial polarity and the pathogenesis of disease states. N. Engl. J. Med. 330:1580-1588.
7. Tsukamoto, T., and Nigam, S. K. 1997. Tight junction proteins form large complexes and associate with the cytoskeleton in an ATP depletion model for reversible junction assembly. J. Biol. Chem. 272:16133-16139.
8. Bush, K. T., Tsukamoto, T., and Nigam, S. K. 2000. Selective degradation of E-cadherin and dissolution of E-cadherin-catenin complexes in epithelial ischemia. Am. J. Physiol. Renal Physiol. 278:F847-F852.
9. Bush, K. T., George, S. K., Zhang, P. L., and Nigam, S. K. 1999. Pretreatment with inducers of ER molecular chaperones protects epithelial cells subjected to ATP depletion. Am. J. Physiol. Renal Physiol. 277:F211-F218.
10. Hammerman, M., Safirstein, R., Harris, R., Toback, F., and Humes, H. 2000. Acute renal failure. III. The role of growth factors in the process of renal regeneration and repair. Am. J. Physiol. Renal Physiol. 279:F3-F11.
11. Gailit, J., Colflesh, D., Rabiner, I., Simone, J., and Goligorsky, M. S. 1993. Redistribution and dysfunction of integrins in cultured renal epithelial cells exposed to oxidative stress. Am. J. Physiol. Renal Physiol. 264:F149-F157.
12. Lieberthal, W. et al. 1997. Beta1 integrin-mediated adhesion between renal tubular cells after anoxic injury. J. Am. Soc. Nephrol. 8:175-183.
13. Zuk, A., Bonventre, J. V., Brown, D., and Matlin, K. S. 1998. Polarity, integrin, and extracellular matrix dynamics in the postischemic rat kidney. Am. J. Physiol. Renal Physiol. 275:C711-C731.
14. Gumbiner, B., Stevenson, B., and Grimaldi, A. 1988. The role of the cell adhesion molecule uvomorulin in the formation and maintenance of the epithelial junctional complex. J. Cell Biol. 107:1575-1587.
15. McNeill, H., Ozawa, M., Kemler, R., and Nelson, W. J. 1990. Novel function of the cell adhesion molecule uvomorulin as an inducer of cell surface polarity. Cell. 62:309-316.
16. Steinberg, M. S., and McNutt, P. M. 1999. Cadherins and their connections: adhesion junctions have broader functions. Curr. Opin. Cell Biol. 11:554-560.
17. Mandel, L. J., Doctor, R. B., and Bacallao, R. 1994. ATP depletion: a novel method to study junctional properties in epithelial tissues. II. Internalization of Na⁺, K(+)-ATPase and E-cadherin. J. Cell Sci. 107:3315-3324.
18. Le, T. L., Yap, A. S., and Stow, J. L. 1999. Recycling of E-cadherin: a potential mechanism for regulating cadherin dynamics. J. Cell Biol. 146:219-232.
19. Tsukita, S., Furuse, M., and Itoh, M. 1999. Structural and signalling molecules come together at tight junctions. Curr. Opin. Cell Biol. 11:628-633.
20. Denker, B. M., and Nigam, S. K. 1998. Molecular structure and assembly of the tight junction. Am. J. Physiol. Renal Physiol. 274:F1-F9.
21. Gopalakrishnan, S., Raman, N., Atkinson, S. J., and Marrs, J. A. 1998. Rho GTPase signaling regulates tight junction assembly and protects tight junctions during ATP depletion. Am. J. Physiol. Cell. Physiol. 275:C798-C809.
22. Tsukamoto, T., and Nigam, S. K. 1999. Role of tyrosine phosphorylation in the reassembly of occludin and other tight junction proteins. Am. J. Physiol. Renal Physiol. 276:F737-F750.
23. Ye, J., Tsukamoto, T., Sun, A., and Nigam, S. K. 1999. A role for intracellular calcium in tight junction reassembly after ATP depletion-repletion. Am. J. Physiol. Renal Physiol. 277:F524-F532.
24. Kuznetsov, G., and Nigam, S. K. 1998. Folding of secretory and membrane proteins. N. Engl. J. Med. 339:1688-1695.
25. Van Why, S. K. et al. 1999. Thresholds for cellular disruption and activation of the stress response in renal epithelia. Am. J. Physiol. Renal Physiol. 277:F227-F234.
26. Gething, M. J., and Sambrook, J. 1992. Protein folding in the cell. Nature. 355:33-45.
27. Nigam, S. K. et al. 1994. A set of endoplasmic reticulum proteins possessing properties of molecular chaperones includes Ca(2+)-binding proteins and members of the thioredoxin superfamily. J. Biol. Chem. 269:1744-1749.
28. Gabai, V. L., and Kabakov, A. E. 1993. Rise in heat-shock protein level confers tolerance to energy deprivation. FEBS Lett. 327:247-250.
29. Georgopoulos, C., and Welch, W. J. 1993. Role of the major heat shock proteins as molecular chaperones. Annu. Rev. Cell Biol. 9:601-634.
30. Yoo, C. G. et al. 2000. Anti-inflammatory effect of heat shock protein induction is related to stabilization of I kappa B alpha through preventing I kappa B kinase activation in respiratory epithelial cells. J. Immunol. 164:5416-5423.
31. Bush, K. T., Goldberg, A. L., and Nigam, S. K. 1997. Proteasome inhibition leads to a heat-shock response, induction of endoplasmic reticulum chaperones, and thermotolerance. J. Biol. Chem. 272:9086-9092.
32. Dong, Z., Saikumar, P., Griess, G. A., Weinberg, J. M., and Venkatachalam, M. A. 1998. Intracellular Ca2+ thresholds that determine survival or death of energy-deprived cells. Am. J. Pathol. 152:231-240.
33. Kribben, A. et al. 1994. Evidence for role of cytosolic free calcium in hypoxia-induced proximal tubule injury. J. Clin. Invest. 93:1922-1929.
34. Liu, H., Miller, E., van de Water, B., and Stevens, J. L. 1998. Endoplasmic reticulum stress proteins block oxidant-induced Ca2+ increases and cell death. J. Biol. Chem. 273:12858-12862.
35. Yu, Z., Luo, H., Fu, W., and Mattson, M. P. 1999. The endoplasmic reticulum stress-responsive protein GRP78 protects neurons against excitotoxicity and apoptosis: suppression of oxidative stress and stabilization of calcium homeostasis. Exp. Neurol. 155:302-314.
36. Bian, X., Hughes, F. M. Jr., Huang, Y., Cidlowski, J. A., and Putney, J. W. Jr. 1997. Roles of cytoplasmic Ca2+ and intracellular Ca2+ stores in induction and suppression of apoptosis in S49 cells. Am. J. Physiol. Cell. Physiol. 272:C1241-C1249.

1-6. (canceled)

7. A method for treating an ischemic lesion comprising epithelial cells, the method comprising co-administering to the lesion:
(i) a first composition comprising an agent selected from the group consisting of pleiotrophin, insulin-like growth factors, midkine, fibroblast growth factors, epidermal growth factor receptor ligands, melanocyte stimulating hormone, and hepatocyte growth factor;
(ii) a second composition comprising tunicamycin or geldanomycin; and
(iii) optionally a third composition comprising MG132 or lactocystin.

8. The method of claim 7, wherein the first composition comprises pleiotrophin and the second composition comprises tunicamycin

9. A method for preventing or inhibiting tissue damage associated with ischemia, the method comprising:
(i) identifying an ischemic lesion comprising epithelial cells
(ii) co-administering to the lesion:
a) at least one composition comprising an agent that enhances protein folding and assembly capacity in the ER and/or cytosol;
b) at least one composition comprising an agent that inhibits degradation of proteins necessary for the maintenance of the polarized epithelial cell phenotype; and
c) optionally at least one composition comprising an agent that inhibits internalization of one or more intercellular junction proteins.

10. The method of claim 9 further including administering at least one composition comprising an agent that promotes activation of specific signaling events during short-term ischemia.

11. The method according to claim 10, wherein the promoting refers to facilitating the resorting of growth factor receptors to the cell surface thereby enhancing the effectiveness of endogenous and/or exogenous growth factors administered after ischemic insult.

12. The method according to claim 9, wherein the inhibiting of the internalization comprises contacting the lesion with drugs or growth factors that specifically modulate signaling through a mechanism selected from the group consisting of IP₃-sensitive calcium stores, G-proteins, protein kinase C, and other kinases implicated in reassembly response during calcium switch.

13. The method according to claim 9, wherein the inhibiting degradation refers to prevention of proteolytic cleavage of proteins.

14. The method according to claim 13, wherein the prevention of proteolytic cleavage comprises proteasome inhibition.

15. The method according to claim 9, wherein at least one composition comprising an agent that enhances protein folding and assembly capacity in the ER and/or cytosol comprises tunicamycin.

16. The method of claim 9, wherein intracellular membrane proteins are E-cadherin, claudin and/or occluding.

17. The method of claim 9, wherein the agent is selected from the group consisting of a growth factor, a protein kinase C activator, a GTP binding protein activator, a proteasome inhibitor, a caspase inhibitor, an agent that upregulates cytoprotective chaperones, and an agent that modulates stress responses.

18. The method of claim 17, wherein the proteosome inhibitor is MG132 and/or lactocystin.

19. The method of claim 17, wherein the agent that upregulates cytoprotective chaperones is MG132 and/or lactocystin.

20. The method of claim 17, wherein the growth factor is selected from the group consisting of insulin-like growth factor, pleiotrophin, midkine, fibroblast growth factor, epidermal growth factor receptor ligands, melanocyte stimulating hormone, hepatocyte growth factor.

21. The method of claim 17, wherein the protein kinase C activator is a diacylglycerol analog.

22. The method of claim 17, wherein the GTP binding protein activator is selected from the group consisting of a nonhydrolyzable GTP analog, aluminum fluoride, lysophosphatidic acid and phenylphrine.

23. The method of claim 17, wherein the agent that modulates stress responses is selected form the group consisting of tunicamycin and geldanomycin.
